(19) United States
(12) Patent Application Publication
(10) Pub. No.: US 2006/0288454 A1
(43) Pub. Date: Dec. 21, 2006

(54) Plants having modified growth characteristics and method for making the same

(76) Inventor: Ana Isabel Sanz Molinero, Gentbrugge (BE)

Correspondence Address: NIXON & VANDERHYE, PC, 901 NORTH GLEBE ROAD, 11TH FLOOR, ARLINGTON, VA, 22203, US

(21) Appl. No.: 10/553,656

(22) PCT Filed: Apr. 14, 2004
(86) PCT No.: PCT/EP04/50519

§ 371 Date: Oct. 14, 2005

(30) Foreign Application Priority Data
Apr. 14, 2003 (EP) 03076086.2

(51) Int. Cl. A01H 5/00; C12N 15/82; C12N 5/04; A01H 1/00

(52) U.S. Cl. 800/287; 800/306; 435/419; 435/468

(57) ABSTRACT

The present invention concerns a method for modifying the growth characteristics of plants by modulating expression in a plant of a nucleic acid sequence encoding a metallothionein and/or modulating activity in a plant of a metallothionein. The invention also relates to transgenic plants having modified growth characteristics, which plants have modulated expression of a nucleic acid encoding a metallothionein.

### FIELD OF THE INVENTION

The present invention concerns a method for modifying plant growth characteristics. More specifically, the present invention concerns a method for modifying plant growth characteristics, comprising introduction into a plant of a nucleic acid encoding a metallothionein protein. The present invention also concerns plants having modulated expression of a nucleic acid sequence encoding a metallothionein and/or modulated activity of a metallothionein, which plants have modified growth characteristics relative to corresponding wild type plants. The invention also provides constructs suitable for use in the methods of the invention.

### BACKGROUND OF THE INVENTION

Some heavy metals, particularly copper and zinc, are essential micronutrients that play a role in a range of plant physiological processes via the action of Cu- and Zn-dependent enzymes. These and other nonessential heavy metal ions, such as cadmium, lead, and mercury, are highly reactive and consequently can be toxic to living cells. Thus plants, like all living organisms, have evolved a suite of mechanisms that control and respond to the uptake and accumulation of both essential and nonessential heavy metals. These mechanisms include the chelation and sequestration of heavy metals by particular ligands. The two best-characterized heavy metal-binding ligands in plant cells are the phytochelatins (PCs) and metallothioneins (MTs). MTs are cysteine-rich polypeptides encoded by a family of genes. In contrast, PCs are a family of enzymatically synthesized cysteine-rich peptides.

Metallothioneins, products of mRNA translation, are low molecular weight, cysteine-rich, metal-binding proteins. MT proteins and genes are found throughout the animal and plant kingdoms as well as in the prokaryote Synechococcus. The large number of cysteine residues in MTs bind a variety of metals by mercaptide bonds. MTs typically contain two metal-binding, cysteine-rich domains that give these metalloproteins a dumbbell conformation. The data available today tends to support a role for MTs in copper tolerance (as PCs protect against cadmium).

The classification of MT proteins is based on the arrangement of Cys residues. Cobbett and Goldsbrough (Annu Rev Plant Biol. 53, 159-82, 2002) discriminate four classes: Type 1 to Type 4. Type 2 MTs contain two cysteine rich domains separated by a spacer of approximately 40 amino acids, with the first pair of cysteines present as a Cys-Cys motif in amino acid positions 3 and 4. In addition, the sequences of the N-terminal domain of Type 2 MTs (MSCCGGNCGCS-) are highly conserved.

Arabidopsis, rice and sugarcane contain genes encoding all four types of MTs. General observations may be made about the expression pattern of these genes. Type 4 MTs are restricted to developing seeds. Type 1 MT expression tends to be higher in roots than shoots, whereas generally the reverse is observed for Type 2 MTs. Type 3 MTs are expressed in fleshy fruits or in leaves of non-fleshy fruit producing plants (like Arabidopsis). Various environmental factors influence the expression of these genes.

Transgenics plants expressing GUS under the control of the AtMT2a promoter have been produced. In young plants, staining was found only in cotyledons and lateral root tips. GUS activity in older plants was seen at the base of trichomes, in hydathodes, sepals, anthers, stigma, root tips, and vascular tissues at branch points of lateral roots. As leaves aged, GUS staining increased.

Transgenic plants expressing MTs have been produced serving two purposes:
1) Phytoremediation of heavy metal contaminated soils: transgenic tobacco and Brassica plants, engineered to overexpress MTs, moderately resist the toxicity of heavy metals like cadmium (Suh et al., Mol Cells. 8, 678-684, 1998).
2) Nutritional quality improvement, when co-expressed in rice with phytase and ferritin, to improve iron diet in humans. The metallothionein-like protein, rich in cysteine, (a sulfur-rich amino acid) helps in iron absorption by the human digestive system (Potrykus I (2002) Nutritional improvement of rice to reduce malnutrition in developing countries. In “Plant Biotechnology 2002 and Beyond” I. K. Vasil (ed.) pp 401-406).

Given the ever-increasing world population, it remains a major goal of agricultural research to improve the efficiency of agriculture and to increase the diversity of plants in horticulture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic complements that may not always result in the desirable trait being passed on from parent plants. Advances in molecular biology have allowed mankind to manipulate the germplasm of animals and plants. Genetic engineering of plants entails the isolation and manipulation of genetic material (typically in the form of DNA or RNA) and the subsequent introduction of that genetic material into a plant. Such technology has led to the development of plants having various improved economic, agronomic or horticultural traits. A trait of particular economic interest is high yield.

### DETAILED DESCRIPTION

It has now surprisingly been found that modulating expression of a nucleic acid encoding a metallothionein protein in a plant gives rise to plants having modified growth and development. Therefore according to a first embodiment of the present invention there is provided a method for modifying plant growth and development, comprising introducing a genetic modification in a plant and selecting for modulated expression in a plant of a nucleic acid encoding a metallothionein protein, provided that the modified growth and development is not increased metal accumulation or increased tolerance or resistance to abiotic stress. With increased metal accumulation is meant any uptake of metals for the purpose of bioremediation or for improving the nutritional quality of plants. In particular, the uptake of heavy metals or iron is envisaged. The term abiotic stress stands for stresses caused by salt, cold or osmotic pressure and includes also oxidative stress.

The methods of the present invention encompass a genetic modification of a plant or a plant cell. The term “genetic modification” refers to a change by human intervention in the genetic content of a cell compared to a wild type cell and includes techniques like genetic engineering, breeding or mutagenesis. The change in genetic content comprises modifications of the genome and includes addition, deletion and substitution of genetic material in the chromosomes of a plant cell as well as in episomes. The term also encompasses the addition of extrachromosomal information to a plant cell. Preferably, the genetic modification results in modulated expression of a nucleic acid. The methods of the present invention also encompass a subsequent step of selection, during which plants with the desired characteristics are selected. The selection step may be based on monitoring the presence or absence of modified growth characteristics, or on monitoring the presence or absence of selectable or screenable marker genes linked an introduced nucleic acid of interest.

In the present invention modulated expression, and in particular increased expression, of a nucleic acid is envisaged. Modulating expression (increasing or decreasing expression) of a nucleic acid encoding a metallothionein or modulation of the activity and/or levels of the metallothionein itself encompasses altered expression of a gene and/or altered activity and/or levels of a gene product, namely a polypeptide, in specific cells or tissues. Altered expression of a gene and/or altered activity and/or levels of a gene product may be effected, for example by chemical means and/or recombinant means. Modulating expression of a gene and/or levels of a gene product and/or modulating activity of a gene product may be effected directly through the modulation of expression of a metallothionein-encoding gene and/or directly through the modulation of the activity and/or levels of a metallothionein protein. The modulated expression may result from altered expression levels of an endogenous metallothionein gene and/or may result from altered expression of a metallothionein encoding nucleic acid that was previously introduced into a plant. Similarly, modulated levels and/or activity of a metallothionein protein may be the result of altered expression levels of an endogenous metallothionein gene and/or may result from altered expression of a metallothionein encoding nucleic acid that was previously introduced into a plant. Additionally or alternatively, the modulation of expression as mentioned above is effected in an indirect way, for example may be effected as a result of decreased or increased levels and or activity of factors that control the expression of a metallothionein gene or that influence the activity and/or levels of the metallothionein.

Advantageously, modulation of expression of a nucleic acid encoding a metallothionein and/or modulation of activity and/or levels of the metallothionein itself may be effected by chemical means, i.e. by exogenous application of one or more compounds or elements capable of modulating activity and/or levels of the metallothionein protein and/or capable of modulating expression of a metallothionein encoding nucleic acid (which nucleic acid may be either an endogenous gene or a transgene introduced into a plant). The term “exogenous application” taken in its broadest context includes contacting or administering cells, tissues, organs or organisms with a suitable compound or element. The compound may be applied to a plant in a suitable form for uptake (such as through application to the soil for uptake via the roots, or by applying directly to the leaves, for example by spraying).

Suitable compounds or elements for exogenous application include metallothionein encoding nucleic acids and nucleic acids that hybridise therewith; the metallothionein gene product or a homologue, derivative or active fragment thereof and/or antibodies recognizing or mimicking the gene product. Such antibodies may comprise “plantibodies”, single chain antibodies, IgG antibodies and heavy chain antibodies from camels or other members of the Camelidae, as well as fragments thereof. Additionally or alternatively, contacting or administering cells, tissues, organs or organisms with an interacting protein or with an inhibitor or activator of the gene/gene product provides another exogenous means for modulation of expression of a nucleic acid encoding a metallothionein and/or for modulation of activity and/or level of the metallothionein itself. Modulation of expression of a nucleic acid encoding a metallothionein protein and/or modulation of activity and/or levels of the metallothionein itself may also be effected as a result of altered levels of factors that directly or indirectly activate or inactivate a metallothionein protein.

Plants, seeds or other plant material can also be subjected to treatment with mutagenic substances. Chemical substances effecting mutagenesis comprise N-nitroso-N-ethylurea, ethylene imine, ethyl methanesulphonate or diethyl sulphate. As an alternative, ionising radiation such as γ-rays or X-rays can equally well be used. Methods for introducing mutations and testing the effect of mutations (such as modified protein expression and/or modified protein activity) are known in the art. Encompassed by mutagenesis are methods employing chemical mutagens, as well as physical mutagens, such as radiation.

Any characteristic of the metallothionein can be altered by mutagenesis. For example, the expression level can be increased or decreased, the activity of the protein can be modified, or the metal binding properties can be adapted. According to a preferred aspect of the invention the mutagenesis results in increased expression and/or activity of a metallothionein.

Additionally or alternatively, and according to a preferred embodiment of the present invention, modulation of expression of a nucleic acid sequence encoding a metallothionein and/or modulation of activity of the metallothionein itself may be effected by recombinant means. Such recombinant means may comprise a direct and/or indirect approach for modulation of expression of a nucleic acid sequence and/or for modulation of the activity of a protein.

For example, an indirect approach may comprise introducing into a plant, a nucleic acid sequence capable of modulating activity and/or levels of the protein in question (a metallothionein) and/or expression of the gene in question (a gene encoding a metallothionein). Examples of such nucleic acids to be introduced into a plant are nucleic acids encoding transcription factors, activators or inhibitors that bind to the promoter of the metallothionein gene or that interact with the metallothionein protein. Methods to test these kinds of interaction and to isolate the nucleic acids encoding these interactors are for example yeast one-hybrid or yeast two-hybrid screening. Also encompassed by an indirect approach for modulating activity of a metallothionein and/or expression of a metallothionein gene is the inhibition or stimulation of regulatory sequences that drive expression of the native gene or transgene. Such regulatory sequences may be introduced into a plant. For example, the regulatory sequence introduced into the plant is a promoter, capable of driving the expression of an endogenous metallothionein gene. Furthermore, modulation of the activity of a metallothionein may be effected by altering levels in a plant of a factor able to interact with metallothionein. Such factors may include ligands (regulators, subunits, substrates, targets) of the metallothionein.

A direct and preferred approach on the other hand for modulating expression of a metallothionein gene or modulating the activity and/or levels of a metallothionein protein comprises introducing into a plant a nucleic acid sequence encoding a metallothionein or a homologue, derivative or active fragment thereof. The nucleic acid sequence may be introduced into a plant by, for example, transformation. Therefore, according to a preferred aspect of the present invention, there is provided a method for modifying growth and development of a plant, in particular for increasing plant yield and/or biomass of a plant comprising a genetic modification of the plant, which genetic modification comprises introducing a metallothionein encoding nucleic acid into a plant.

According to a preferred aspect of the present invention, enhanced or increased expression of a nucleic acid is envisaged. Methods for obtaining enhanced or increased expression of genes or gene products are well documented in the art and include, for example, overexpression driven by a (strong) promoter, the use of transcription enhancers or translation enhancers. The term overexpression as used herein means any form of expression that is additional to the original wild-type expression level. Preferably the nucleic acid to be introduced into the plant and/or the nucleic acid that is to be overexpressed in the plants is in a sense direction with respect to the promoter to which it is operably linked. The nucleic acid to be overexpressed encodes a metallothionein, preferably a type 2 metallothionein as defined below, further preferably the nucleic acid sequence encoding the metallothionein is isolated from a plant, preferably from a dicotyledonous plant, preferably of the family Brassicaceae, further preferably the sequence is isolated from Arabidopsis thaliana, most preferably the nucleic acid sequence is as represented by SEQ ID NO: 1 or a portion thereof, or encodes an amino acid sequence as represented by SEQ ID NO: 2 or a homologue, derivative or active fragment thereof. It should be noted that the applicability of the invention does not rest upon the use of the nucleic acid represented by SEQ ID NO: 1, nor upon the nucleic acid sequence encoding the amino acid sequence of SEQ ID NO: 2, but that other nucleic acid sequences encoding homologues, derivatives or active fragments of SEQ ID NO: 2, or portions of SEQ ID NO: 1, or sequences hybridising with SEQ ID NO: 1 may be used in the methods of the present invention. In particular, homologues from other plant species such as tobacco, maize or rice are also useful in the present invention.

Alternatively and/or additionally, increased expression of a metallothionein encoding gene or increased activities and/or levels of a metallothionein protein in a plant cells, is achieved by mutagenesis of the plant cell. For example these mutations can be responsible for the altered control of a metallothionein-encoding gene, resulting in higher expression of the gene. Mutations can also cause conformational changes of the protein, resulting in higher activity and/or levels of the protein.

The term metallothionein includes proteins homologous to the metallothionein as presented in SEQ ID NO 2. Metallothioneins are well known in the art, for a recent overview and classification, see Cobbett and Goldsbrough (2002). Metallothioneins are small proteins with a dumbbell conformation that finds its origin in conserved N-terminal and C-terminal cysteine rich domains which are separated from each other by a region that is variable in length and amino acid composition. Based on the primary structure 4 types of metallothioneins are discriminated, an alignment of various plant metallothioneins is given in FIG. 1. The metallothionein of SEQ ID NO 2 comprises a conserved N-terminal domain typical for type 2 metallothioneins as defined by Cobbett and Goldsbrough (2002), which domain comprises the consensus sequence “MSCCGG (N/S) CGCG (T/S/A) (G/A/S) C (K/Q/S) C”, accordingly, preferred homologues to be used in the methods of the present invention are metallothioneins comprising this conserved domain. Additionally and/or alternatively, the metallothionein homologues have metal binding activity which can be measured in a metal saturation test (Scheuhammer et al., Toxicol. Appl Pharmacol. 82, 417-425, 1986) and/or may function as a redox sensor (Fabisiak et al., Methods Enzymol. 353, 268-281 (2002)).

Methods for the search and identification of metallothionein homologues would be well within the realm of a person skilled in the art. Such methods comprise comparison of the sequences represented by SEQ ID NO 1 or 2, in a computer readable format, with sequences that are available in public databases such as MIPS (http://mips.gsf.de/), GenBank (http://www.ncbi.nlm.nih.gov/Genbank/index.html) or EMBL Nucleotide Sequence Database (http://www.ebi.ac.uk/embl/index.html), using algorithms well known in the art for the alignment or comparison of sequences, such as GAP (Needleman and Wunsch, J. Mol. Biol. 48; 443-453 (1970)), BESTFIT (using the local homology algorithm of Smith and Waterman (Advances in Applied Mathematics 2; 482-489 (1981))), BLAST (Altschul, S. F., Gish, W., Miller, W., Myers, E. W. & Lipman, D. J., J. Mol. Biol. 215:403-410 (1990)), FASTA and TFASTA (W. R. Pearson and D. J. Lipman Proc. Natl. Acad. Sci. USA 85:2444-2448 (1988)). The software for performing BLAST analysis is publicly available through the National Centre for Biotechnology Information. The abovementioned homologues were identified using blast default parameters (BLOSUM62 matrix, gap opening penalty 11 and gap extension penalty 1) and preferably the full length sequences are used for analysis.

“Homologues” of a metallothionein protein encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W. H. Freeman and Company). The homologues useful in the method according to the invention have at least 50% sequence identity or similarity (functional identity) to the unmodified protein, alternatively at least 60% sequence identity or similarity to an unmodified protein, alternatively at least 70% sequence identity or similarity to an unmodified protein. Typically, the homologues have at least 80% sequence identity or similarity to an unmodified protein, preferably at least 85% sequence identity or similarity, further preferably at least 90% sequence identity or similarity to an unmodified protein, most preferably at least 95% sequence identity or similarity to an unmodified protein. Preferred homologues include the proteins comprising the conserved sequence “MSCCGG (N/S) CGCG (T/S/A) (G/A/S) C (K/Q/S) C”, such as SEQ ID NO 4 or GenBank accessions CAA71803, AAP94016, CAA71804, NP_{—}195858, AAM62956, AAB61212, CAA65009, CAA92243.

Homologous proteins can be grouped in “protein families”. A protein family can be defined by functional and sequence similarity analysis, such as, for example, Clustal W. A neighbour-joining tree of the proteins homologous to metallothionein can be generated by the Clustal W program and gives a good overview of its structural and ancestral relationship. In a particular embodiment of the present invention, the metallothionein homologues belong to the type 2 metallothionein protein family (Cobbett and Goldsbrough, 2002). In the Arabidopsis genome two type 2 family members of the metallothionein protein were identified (GenBank accessions AAA50250, NP_{—}195858). Also in other plants such as rice or other monocotyledonous plants, family members of the metallothionein may be identified. Advantageously also these family members are useful in the methods of the present invention.

Two special forms of homology, orthologous and paralogous, are evolutionary concepts used to describe ancestral relationships of genes. The term “paralogous” relates to homologous genes that result from one or more gene duplications within the genome of a species. The term “orthologous” relates to homologous genes in different organisms due to ancestral relationship of these genes. The term “homologues” as used herein also encompasses paralogues and orthologues of the proteins useful in the methods according to the invention.

Orthologous genes can be identified by querying one or more gene databases with a query gene of interest, using for example the BLAST program. The highest-ranking subject genes that result from the search are then again subjected to a BLAST analysis, and only those subject genes that match again with the query gene are retained as true orthologous genes. For example, to find a rice orthologue of an Arabidopsis thaliana gene, one may perform a BLASTN or TBLASTX analysis on a rice database (such as (but not limited to) the Oryza sativa Nipponbare database available at the NCBI (http://www.ncbi.nlm.nih.gov) or the genomic sequences of rice (cultivars indica or japonica)). In a next step, the obtained rice sequences are used in a reverse BLAST analysis using an Arabidopsis database. The results may be further refined when the resulting sequences are analysed with ClustalW and visualised in a neighbour joining tree. The method can be used to identify orthologues from many different species.

“Homologues” of a metallothionein encompass proteins having amino acid substitutions, insertions and/or deletions relative to the unmodified protein. “Substitutional variants” of a protein are those in which at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues, and deletions will range from about 1 to 20 residues. Preferably, amino acid substitutions comprise conservative amino acid substitutions. “Insertional variants” of a protein are those in which one or more amino add residues are introduced into a predetermined site in a protein. Insertions can comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine) 6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope. “Deletion variants” of a protein are characterised by the removal of one or more amino acids from the protein. Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The term “derivatives” refers to peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as presented in SEQ ID NO: 2. “Derivatives” of a metallothionein protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence such as, for example, a reporter molecule which is bound to facilitate its detection, and non-naturally occurring amino acid residues relative to the amino acid sequence of a naturally-occurring protein.

“Active fragments” or “functional fragments” of a metallothionein encompass at least 15, preferably 20, 25, more preferably 30 or more contiguous amino acid residues of a protein, which residues retain similar biological and/or functional activity to the naturally occurring protein. A preferred fragment of a metallothionein protein comprises at least the conserved N-terminal domain specified above.

The term metallothionein encoding nucleic acid/gene, as defined herein, refers to any nucleic acid encoding a metallothionein protein, or the complement thereof. The nucleic acid may be derived (either directly or indirectly (if subsequently modified)) from any source provided that the nucleic acid, when expressed in a plant, leads to modulated expression of a metallothionein nucleic acid/gene or modulated activity and/or levels of a metallothionein. The nucleic acid may be isolated from an eukaryotic source, such as yeast or fungi, plants (including algae) or animals (including humans). This nucleic acid may be substantially modified from its native form in composition and/or genomic environment through deliberate human manipulation. The nucleic acid sequence is preferably a homologous nucleic acid sequence, i.e. a structurally and/or functionally related nucleic acid sequence, preferably obtained from a plant, whether from the same plant species or different The nucleic acid sequence may be isolated from a dicotyledonous plant species, preferably from the family Brassicaceae, further preferably from Arabidopsis thaliana. More preferably, the nucleic acid is as represented by SEQ ID NO: 1 or a portion thereof or a nucleic acid sequence capable of hybridising therewith, which hybridising sequence encodes proteins having metallothionein protein activity, i.e. similar biological activity to that of SEQ ID NO: 1, or it is a nucleic acid encoding an amino acid represented by SEQ ID NO: 2 or encoding a homologue, derivative or active fragment thereof. This term also encompasses variants of the nucleic acid encoding a metallothionein protein due to the degeneracy of the genetic code; allelic variants of the nucleic acid encoding a metallothionein; and different splice variants of the nucleic acid encoding a metallothionein and variants that are interrupted by one or more intervening sequences.

Advantageously, the method according to the present invention may also be practised using portions of a DNA or nucleic acid sequence, which portions encode a peptide that retains metallothionein activity, i.e. a similar biological function to that of SEQ ID NO: 2. Portions of a DNA sequence refer to a piece of DNA derived or prepared from an original (larger) DNA molecule, which DNA portion, when expressed in a plant, gives rise to plants having modified growth characteristics. The portion may comprise many genes, with or without additional control elements, or may contain just spacer sequences etc.

The present invention also encompasses nucleic acid sequences capable of hybridising with a nucleic acid sequence encoding a metallothionein, which nucleic acid sequences may also be useful in practising the methods according to the invention. The term “hybridisation” as defined herein is a process wherein substantially homologous complementary nucleotide sequences anneal to each other. The hybridisation process can occur entirely in solution, i.e. both complementary nucleic acids are in solution. Tools in molecular biology relying on such a process include the polymerase chain reaction (PCR; and all methods based thereon), subtractive hybridisation, random primer extension, nuclease S1 mapping, primer extension, reverse transcription, cDNA synthesis, differential display of RNAs, and DNA sequence determination. The hybridisation process can also occur with one of the complementary nucleic acids immobilised to a matrix such as magnetic beads, Sepharose beads or any other resin. Tools in molecular biology relying on such a process include the isolation of poly (A⁺) mRNA. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). Tools in molecular biology relying on such a process include RNA and DNA gel blot analysis, colony hybridisation, plaque hybridisation, in situ hybridisation and micro array hybridisation. In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration and hybridisation buffer composition.

For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C.

High stringency conditions for hybridisation thus include high temperature and/or low salt concentration (salts include NaCl and Na₃-citrate) but can also be influenced by the inclusion of formamide in the hybridisation buffer and/or lowering the concentration of compounds such as SDS (sodium dodecyl sulphate) in the hybridisation buffer and/or exclusion of compounds such as dextran sulphate or polyethylene glycol (promoting molecular crowding) from the hybridisation buffer. Sufficiently low stringency hybridisation conditions are particularly preferred for the isolation of nucleic acids homologous to the DNA sequences of the invention defined supra. Elements contributing to homology include allelism, degeneration of the genetic code and differences in preferred codon usage.

“Stringent hybridisation conditions” and “stringent hybridisation wash conditions” in the context of nucleic acid hybridisation experiments such as Southern and Northern hybridisations are sequence dependent and are different under different environmental parameters. For example, longer sequences hybridise specifically at higher temperatures. The Tₘ is the temperature under defined ionic strength and pH, at which 50% of the target sequence hybridises to a perfectly matched probe. Specificity is typically the function of post-hybridisation washes. Critical factors of such washes include the ionic strength and temperature of the final wash solution.

Generally, stringent conditions are selected to be about 50° C. lower than the thermal melting point (Tₘ) for the specific sequence at a defined ionic strength and pH. The Tₘ is the temperature under defined ionic strength and pH, at which 50% of the target sequence hybridises to a perfectly matched probe. The Tₘ is dependent upon the solution conditions and the base composition of the probe, and may be calculated using the following equation:
Tₘ=79.8° C.+(18.5×log [Na⁺])+(58.4° C.×% [G+C])−(820×(#bp in duplex)⁻¹)−(0.5×% formamide)

More preferred stringent conditions are when the temperature is 20° C. below Tₘ, and the most preferred stringent conditions are when the temperature is 10° C. below Tₘ. Non-specific binding may also be controlled using any one of a number of known techniques such as, for example, blocking the membrane with protein containing solutions, additions of heterologous RNA, DNA, and SDS to the hybridisation buffer, and treatment with Rnase.

Wash conditions are typically performed at or below hybridisation stringency. Generally, suitable stringent conditions for nucleic acid hybridisation assays or gene amplification detection procedures are as set forth above. More or less stringent conditions may also be selected.

For the purposes of defining the level of stringency, reference can conveniently be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989). An example of low stringency conditions is 4-6×SSC/0.1-0.5% w/v SDS at 37-45° C. for 2-3 hours. Depending on the source and concentration of the nucleic acid involved in the hybridisation, alternative conditions of stringency may be employed such as medium stringent conditions. Examples of medium stringent conditions include 1-4×SSC/0.25% w/v SDS at ≧45° C. for 2-3 hours. An example of high stringency conditions includes 0.1-1×SSC/0.1% w/v SDS at 60° C. for 1-3 hours. The skilled artisan is aware of various parameters which may be altered during hybridisation and washing and which will either maintain or change the stringency conditions. For example, another stringent hybridisation condition is hybridisation at 4×SSC at 65° C., followed by a washing in 0.1×SSC, at 65° C. for about one hour. Alternatively, an exemplary stringent hybridisation condition is in 50% formamide, 4×SSC at 42° C. Still another example of stringent conditions include hybridisation at 62° C. In 6×SSC, 0.05× BLOTTO and washing at 2×SSC, 0.1% w/v SDS at 62° C.

The methods according to the present invention may also be practised using an alternative splice variant of a nucleic acid sequence encoding a metallothionein. The term “alternative splice variant” as used herein encompasses variants of a nucleic acid sequence in which selected introns and/or exons have been excised, replaced or added. Such variants will be ones in which the biological activity of the protein remains unaffected, which can be achieved by selectively retaining functional segments of the protein. Such splice variants may be found in nature or can be manmade. Methods for making such splice variants are well known in the art. Therefore according to another aspect of the present invention, there is provided, a method for modifying the growth characteristics of plants, comprising modulating expression in a plant of an alternative splice variant of a nucleic acid sequence encoding a metallothionein protein and/or by modulating activity and/or levels of a metallothionein protein encoded by the alternative splice variant. Preferably, the splice variant is a splice variant of the sequence represented by SEQ ID NO: 1.

Advantageously, the methods according to the present invention may also be practised using allelic variants of a nucleic acid sequence encoding a metallothionein, preferably an allelic variant of a sequence represented by SEQ ID NO: 1. Allelic variants exist in nature and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp). SNPs and INDELs form the largest set of sequence variants in naturally occurring polymorphic strains of most organisms. They are helpful in mapping genes and discovery of genes and gene functions. They are furthermore helpful in identification of genetic loci, e.g. plant genes, involved in determining processes such as growth rate, plant size and plant yield, plant vigor, disease resistance, stress tolerance etc. Many techniques are nowadays available to identify SNPs and/or INDELs including (i) PCR followed by denaturing high performance liquid chromatography (DHPLC; e.g. Cho et al. (1999) Nature Genet. 23, 203-207); (ii) constant denaturant capillary electrophoresis (CDCE) combined with high-fidelity PCR (e.g. Li-Sucholeiki et al. (1999) Electrophoresis 20, 1224-1232); (iii) denaturing gradient gel electrophoresis (Fischer and Lerman (1983) Proc. Natl. Acad. Sci. USA 80, 1579-1583); (iv) matrix-assisted laser desorption/ionization time-of-flight mass spectrometry (MALDI-TOF MS; e.g. Ross et al. (2000) Biotechniques 29, 620-629); (v) real-time fluorescence monitoring PCR assays (Tapp et al. (2000) Biotechniques 28, 732-738); (vi) AcryditeTM gel technology (Kenney et al. (1998) Biotechniques 25, 516-521); (vii) cycle dideoxy fingerprinting (CddF; Langemeier et al. (1994) Biotechniques 17, 484-490); (viii) single-strand conformation polymorphism (SSCP) analysis (Vidal-Puig and Moller (1994) Biotechniques 17, 490-496) and (ix) mini-sequencing primer extension reaction (Syvanen (1999) Hum. Mutat 13, 1-10). The technique of ‘Targeting Induced Local Lesions in Genomes’ (TILLING; McCallum et al. (2000) Nat. Biotechnol. 18, 455-457; Plant Physiol. 123, 439-442), which is a variant of (i) supra, can also be applied to rapidly identify an altered gene in e.g. chemically mutagenised plant individuals showing interesting phenotypes.

The use of these allelic variants in particular conventional breeding programmes, such as in marker-assisted breeding is also encompassed by the present invention; this may be in addition to their use in the methods according to the present invention. Such breeding programmes sometimes require the introduction of allelic variations in the plants by mutagenic treatment of a plant. One suitable mutagenic method is EMS mutagenesis. Identification of allelic variants then may take place by, for example, PCR. This is followed by a selection step for selection of superior allelic variants of the metallothionein sequence in question and which give rise to modified growth and development in a plant. Selection is typically carried out by monitoring growth performance of plants containing different allelic variants of the sequence in question, for example, different allelic variants of SEQ ID NO: 1. Monitoring growth performance can be done in a greenhouse or in the field. Further optional steps include crossing plants, in which the superior allelic variant was identified, with another plant. This could be used, for example, to make a combination of interesting phenotypic features. Therefore, as mutations in the metallothionein gene may occur naturally, they may form the basis for selection of plants showing higher yield. The present invention thus encompasses the use of a nucleic acid encoding a metallothionein or capable of modulating the activity and/or levels of a metallothionein in breeding programs.

According to another aspect of the present invention, advantage may be taken of the nucleotide sequence capable of modulating expression of a nucleic acid encoding a metallothionein in breeding programmes. The nucleic acid sequence may be on a chromosome, or a part thereof, comprising at least the nucleic acid sequence encoding the metallothionein and preferably also one or more related family members. In an example of such a breeding programme, a DNA marker is identified which may be genetically linked to a gene capable of modulating expression of a nucleic acid encoding a metallothionein in a plant, which gene may be a gene encoding the metallothionein itself or any other gene which may directly or indirectly influence expression of the gene encoding a metallothionein and/or activity of the metallothionein itself. This DNA marker may then used in breeding programs to select plants having altered growth characteristics.

The methods according to the present invention may also be practised by introducing into a plant at least a part of a (natural or artificial) chromosome (such as a Bacterial Artificial Chromosome (BAC)), which chromosome contains at least a gene/nucleic acid sequence encoding a metallothionein (such as SEQ ID NO: 1 or SEQ ID NO: 3), preferably together with one or more related gene family members. Therefore, according to a further aspect of the present invention, there is provided a method for modifying the growth and development of plants by introducing into a plant at least a part of a chromosome comprising at least a gene/nucleic acid encoding a metallothionein.

Accordingly, as another aspect of the invention, there is provided a method for modifying growth and development, preferably for increasing plant yield and/or biomass, comprising modulating expression, preferably increasing expression in a plant of a nucleic acid sequence encoding a metallothionein and/or modulating activity and/or levels in a plant of a metallothionein, wherein said nucleic acid sequence and said proteins include variants chosen from:
(i) a nucleic acid as represented by SEQ ID NO: 1 or encoding a metallothionein protein as represented by SEQ ID NO: 2;
(ii) an alternative splice variant of a nucleic acid sequence encoding a metallothionein protein or wherein said metallothionein protein is encoded by a splice variant;
(iii) an allelic variant of a nucleic acid sequence encoding a metallothionein protein or wherein said metallothionein protein is encoded by an allelic variant;
(iv) a nucleic acid encoding a functional portion of a metallothionein protein;
(v) a metallothionein protein as represented by SEQ ID NO: 2
(vi) homologues, derivatives and active fragments of a metallothionein protein.

According to one aspect of the present invention, enhanced or increased expression of a nucleic acid is envisaged. Methods for obtaining enhanced or increased expression of genes or gene products are well documented in the art and include, for example, overexpression driven by a (strong) promoter, the use of transcription enhancers or translation enhancers. The term overexpression as used herein means any form of expression that is additional to the original wild-type expression level. Preferably, the nucleic acid to be introduced into the plant and/or to be overexpressed is oriented in sense direction with respect to the promoter to which it is operably linked. Preferably, the nucleic acid to be overexpressed encodes a metallothionein, further preferably the nucleic acid sequence encoding the metallothionein is isolated from a dicotyledonous plant, preferably of the family Brassicaceae, further preferably wherein the sequence is isolated from Arabidopsis thaliana, most preferably the nucleic acid sequence is as represented by SEQ ID NO: 1 or a portion thereof, or encodes an amino acid sequence as represented by SEQ ID NO: 2 or a homologue, derivative or active fragment thereof. Alternatively, the nucleic acid sequence encoding the metallothionein is as represented by SEQ ID NO: 3 or is a portion thereof, or encodes an amino acid sequence as represented by SEQ ID NO: 4 or encodes a homologue, derivative or active fragment thereof. It should be noted that the applicability of the invention is not limited to use of the nucleic acid represented by SEQ ID NO: 1 nor to the nucleic acid sequence encoding the amino acid sequence of SEQ ID NO: 2, but that other nucleic acid sequences encoding homologues, derivatives or active fragments of SEQ ID NO: 2, or portions of SEQ ID NO: 1, or sequences hybridising with SEQ ID NO: 1 may be used in the methods of the present invention.

According to another aspect of the present invention, decreased expression of a nucleic acid sequence is envisaged. Modulating gene expression (whether by a direct or indirect approach) encompasses altered transcript levels of a gene. Altered transcript levels can be sufficient to induce certain phenotypic effects, for example via the mechanism of cosuppression. Here the overall effect of overexpression of a transgene is that there is less activity in the cell of the protein encoded by a native gene having homology to the introduced transgene. Other examples of decreasing expression are also well documented in the art and include, for example, downregulation of expression by anti-sense techniques, co-suppression techniques, RNAi techniques, small interference RNAs (siRNAs), microRNA (miRNA), the use of ribozymes, etc. Therefore according to a particular aspect of the invention, there is provided a method for modulating growth characteristics of plants, including technologies that are based on the synthesis of antisense transcripts, complementary to the mRNA of a metallothionein gene, or based on RNA interference. Advantageously, the methods according to the present invention may also be practised by downregulation of a nucleic acid sequence encoding a metallothionein protein. Plants having modified growth characteristics may be obtained by expressing a nucleic acid sequence encoding a metallothionein in either sense or antisense orientation. Techniques for downregulation are well known in the art. The terms “gene silencing” or “downregulation” of expression, as used herein, refer to lowering levels of gene expression and/or levels of active gene product and/or levels of gene product activity. Such decreases in expression may be accomplished by, for example, the addition of coding sequences or parts thereof in a sense orientation (if it is desired to achieve co-suppression). Therefore, according to one aspect of the present invention, the growth of a plant may be modified by introducing into a plant an additional copy (in full or in part) of a metallothionein gene already present in a host plant The additional gene will silence the endogenous gene, giving rise to a phenomenon known as co-suppression.

Genetic constructs aimed at silencing gene expression may comprise the metallothionein nucleotide sequence, for example as represented by SEQ ID NO: 1 (or one or more portions thereof) in a sense and/or antisense orientation relative to the promoter sequence. The sense or antisense copies of at least part of the endogenous gene in the form of direct or inverted repeats may be utilised in the methods according to the invention. The growth characteristics of plants may also be modified by introducing into a plant at least part of an antisense version of the nucleotide sequence represented, for example, by SEQ ID NO: 1. It should be clear that part of the nucleic acid (a portion) could achieve the desired result. Homologous anti-sense genes are preferred to heterologous ant-sense genes, homologous genes being plant genes, preferably plant genes from the same plant species, and heterologous genes being genes from non-plant species.

Another method for downregulation of gene expression or gene silencing comprises use of ribozymes, for example as described in Atkins et al. 1994 (WO 94/00012), Lenee et al. 1995 (WO 95103404), Lutziger et al. 2000 (WO 00/00619), Prinsen et al. 1997 (WO 97/3865) and Scott et al. 1997 (WO 97/38116).

Gene silencing may also be achieved by insertion mutagenesis (for example, T-DNA insertion or transposon insertion) or by gene silencing strategies as described by, among others, Angell and Baulcombe 1998 (WO 98/36083), Lowe et al. 1989 (WO 98/53083), Lederer et al. 1999 (WO 99/15682) or Wang et al. 1999 (WO 99/53050). Expression of an endogenous gene may also be reduced if the endogenous gene contains a mutation. Such a mutant gene may be isolated and introduced into the same or different plant species in order to obtain plants having modified growth characteristics.

Advantageously, performance of the method according to the present invention results in plants having a variety of modified growth characteristics, such modified growth characteristics including modified growth, modified yield/biomass, modified architecture and a modified cell division, each relative to corresponding wild type plants. Preferably, the modified growth characteristics are improved growth characteristics and include modified architecture, increased yield/biomass with the proviso that increased yield is not increased metal accumulation, modified stress response with the proviso that the stress is not abiotic stress, and faster growth, each relative to corresponding wild type plants.

The present invention relates to methods to alter growth characteristics of a plant or to methods to produce plants with altered growth characteristics, wherein the growth characteristics comprise any one or more selected from: increased yield, increased biomass, increased total above ground area, increased plant height, increased number of tillers, increased number of primary panicles, increased number of secondary panicles, increased total number of seeds, increased number of filled seeds, increased total seed yield per plant, increased harvest index, increased thousand kernel weight, increased Tmid, increased T45 or A90, increased A42, altered cycling time and/or an altered growth curve. The present invention also provides methods to alter one of the above mentioned growth characteristics, without causing a penalty on one of the other growth characteristics, for example increase of the above ground green tissue area while retaining the same number of filled seeds and the same seed yield.

The term “increased yield” encompasses an increase in biomass in one or more parts of a plant relative to the biomass of corresponding wild-type plants. The term also encompasses an increase in seed yield, which includes an increase in the biomass of the seed (seed weight) and/or an increase in the number of (filled) seeds and/or in the size of the seeds and/or an increase in seed volume, each relative to corresponding wild-type plants. For maize, the increase of seed yield may be reflected in an increase of rows (of seeds) per ear and/or an increased number of kernels per row. An increase in seed size and/or volume may also influence the composition of seeds. An increase in seed yield could be due to an increase in the number and/or size of flowers. An increase in yield might also increase the harvest index, which is expressed as a ratio of the total biomass over the yield of harvestable parts, such as seeds; or thousand kernel weight. Increased yield also encompasses the capacity for planting at higher density (number of plants per hectare or acre).

Also modified cell division may contribute to yield increase. The term “modified cell division” encompasses an increase or decrease in cell division or an abnormal cell division/cytokinesis, altered plane of division, altered cell polarity, altered cell differentiation. The term also comprises phenomena such as endomitosis, acytokinesis, polyploidy, polyteny and endoreduplication. It can be envisaged that plants having increased biomass and height exhibit a modified growth rate when compared to corresponding wild-type plants. The term “modified growth rate” as used herein encompasses, but is not limited to, a faster rate of growth in one or more parts of a plant (including green biomass and including seeds), at one or more stages in the life cycle of a plant. The term “modified growth” encompasses enhanced vigour, earlier flowering, modified cycling time. Plants with modified growth may show a modified growth curve and may have modified values for their Tmid or T90 (respectively the time needed to reach half of their maximal area or 90% of their area, each relative to corresponding wild-type plants).

According to a preferred feature of the present invention, performance of the methods according to the present invention result in plants having increased yield and/or increased biomass, in particular, performance of the methods of the present invention result in plants having increased seed yield. Preferably, the increased yield includes an increased total number of seeds and/or increased total weight of seeds, each relative to control plants. Therefore, according to the present invention, there is provided a method for increasing yield of plants, which method comprises modulating expression of a nucleic add sequence encoding a metallothionein and/or modulating activity of the metallothionein itself in a plant, preferably wherein the metallothionein is encoded by a nucleic add sequence represented by SEQ ID NO: 1 or a portion thereof or sequences capable of hybridising therewith or wherein the metallothionein is represented by SEQ ID NO: 2 or a homologue, derivative or active fragment thereof. Alternatively, the metallothionein may be encoded by a nucleic acid sequence represented by SEQ ID NO: 3, or by a portion thereof or by sequences capable of hybridising therewith, or wherein the metallothionein is represented by SEQ ID NO: 4, or a homologue, derivative or active fragment of any thereof.

“Modified architecture” may be due to change in cell division. The term “architecture” as used herein encompasses the appearance or morphology of a plant, including any one or more structural features or combination of structural features thereof. Such structural features include the shape, size, number, position, texture, arrangement, and pattern of any cell, tissue or organ or groups of cells, tissues or organs of a plant, including the root, leaf, shoot, stem or tiller, petiole, trichome, flower, inflorescence (for monocotyledonous and dicotyledonous plants), panicles, petal, stigma, style, stamen, pollen, ovule, seed, embryo, endosperm, seed coat, aleurone, fibre, cambium, wood, heartwood, parenchyma, aerenchyma, sieve element, phloem or vascular tissue, amongst others. Modified architecture therefore includes all aspects of modified growth of the plant.

Preferably, the modified architecture includes modified number of primary panicles, modified plant height and modified total area, each relative to control plants. Therefore, according to the present invention, there is provided a method for modifying the architecture of plants, particularly the number of primary panicles, plant height and plant area, which method comprises modulating expression of a nucleic acid sequence encoding a metallothionein and/or modulating activity of the metallothionein itself in a plant, preferably wherein the metallothionein is encoded by a nucleic acid sequence represented by SEQ ID NO: 1 or a portion thereof or sequences capable of hybridising therewith or wherein the metallothionein is represented by SEQ ID NO: 2 or a homologue, derivative or active fragment thereof. Alternatively, the metallothionein may be encoded by a nucleic acid sequence represented by SEQ ID NO: 3, or by a portion thereof or by sequences capable of hybridising with the aforementioned sequences, or wherein the metallothionein is represented by SEQ ID NO: 4, or a homologue, derivative or active fragment thereof.

According to a second embodiment of the present invention, genetic constructs and vectors to facilitate introduction and/or expression of the nucleotide sequences useful in the methods according to the invention are provided. Therefore, according to a second embodiment of the present invention, there is provided a gene construct comprising:
(i) a nucleic acid sequence encoding a metallothionein;
(ii) a GOS2 promoter for driving expression of the nucleic acid sequence of (i); and optionally
(iii) a transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more control sequences allowing expression in prokaryotic and/or eukaryotic host cells.

According to a preferred embodiment of the invention, the genetic construct is an expression vector designed to overexpress the nucleic acid sequence. The nucleic acid sequence capable of modulating expression of a nucleic acid encoding a metallothionein and/or activity of the metallothionein itself may be a nucleic acid sequence encoding a metallothionein or a homologue, derivative or active fragment thereof, such as any of the nucleic acid sequences described hereinbefore. A preferred nucleic add sequence is the sequence represented by SEQ ID NO: 1 or a portion thereof or sequences capable of hybridising therewith or a nucleic acid sequence encoding a sequence represented by SEQ ID NO: 2 or a homologue, derivative or active fragment thereof. Preferably, this nucleic acid is cloned in the sense orientation relative to the control sequence to which it is operably linked.

Plants are transformed with a vector comprising the sequence of interest (i.e., the nucleic add sequence capable of modulating expression of a nucleic acid encoding a metallothionein), which sequence is operably linked to one or more control sequences (at least a promoter). The terms “regulatory element”, “control sequence” and “promoter” are all used herein interchangeably and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative which confers, activates or enhances expression of a nucleic acid molecule in a cell, tissue or organ. The terms “control sequence”, “regulatory sequence”, “regulatory element” and “promoter” are used interchangeably herein. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence depending on the desired outcome. Preferably, the nucleic acid sequence capable of modulating expression of a gene encoding a metallothionein is operably linked to a constitutive promoter. The term “constitutive” as defined herein refers to a promoter that is expressed predominantly in at least one tissue or organ and predominantly at any life stage of the plant. Preferably the promoter is expressed predominantly throughout the plant. Preferably, the constitutive promoter is the GOS2 promoter from rice, or a promoter of similar strength and/or a promoter with a similar expression pattern. Alternatively, tissue specific promoters may be used. For example, in cases where increased seed yield is envisaged, the use of seed preferred, flower preferred, meristem preferred promoters or promoters active in dividing cells can be contemplated. Promoter strength and/or expression pattern can be analysed for example by coupling the promoter to a reporter gene and assay the expression of the reporter gene in various tissues of the plant. One suitable reporter gene well known to a person skilled in the art is beta-glucuronidase.

Examples of alternative constitutive promoters are presented in Table 1, and these promoters or derivatives thereof are useful for the methods of the present invention.

TABLE 1
Exemplary constitutive promoters for use in
the performance of the present invention

| Gene source | Reference |
|---|---|
| Actin | McElroy et al, Plant Cell, 2: 163-171, 1990 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992 |
| ubiquitin | Christensen et al, Plant Mol. Biol. 18: 675-689, 1992 |
| rice | Buchholz et al, Plant Mol Biol. 25(5): 837-43, 1994 |
| cyclophilin | |
| maize | Lepetit et al, Mol. Gen. Genet. 231: 276-285, 1992 |
| H3 histone | |
| actin 2 | An et al, Plant J. 10(1); 107-121, 1996 |

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art

The genetic constructs of the invention may further include an origin of replication sequence which is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptll that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

In a preferred embodiment, the genetic construct as mentioned above, comprises a metallothionein gene in sense orientation coupled to a promoter that is preferably a constitutive promoter, such as for example the rice GOS2 promoter. Therefore, another aspect of the present invention is a vector construct comprising an expression cassette to SEQ ID NO 7, which cassette comprises the rice GOS2 promoter, the Arabidopsis metallothionein gene presented in SEQ ID NO 2 and the T-zein+T-rubisco deltaGA transcription terminator sequence.

Alternatively, the present invention provides a vector construct comprising an expression cassette essentially similar to SEQ ID NO 7. A sequence essentially similar to SEQ ID NO 7 encompasses a first nucleic acid sequence encoding a protein homologous to SEQ ID NO 2 or hybridising to SEQ ID NO 1, which first nucleic acid is operably linked to a rice GOS2 promoter or a promoter with a similar expression pattern and/or which first nucleic acid is linked to a transcription termination sequence. Therefore according to another aspect of the invention, there is provided a gene construct essentially similar to SEQ ID NO 7, comprising an expression cassette in which is located a nucleic acid sequence encoding a metallothionein protein, chosen from the group comprising:
(i) a nucleic acid sequence represented by SEQ ID NO: 1 or the complement strand thereof;
(ii) a nucleic acid sequence encoding an amino acid sequence represented by SEQ ID NO: 2 or homologues, derivatives or active fragments thereof;
(iii) a nucleic acid sequence according to (i) to (ii) above which is degenerate as a results of the genetic code;
(iv) nucleic acid sequence which is an allelic variant of the nucleic acid sequences according to (i) to (iii);
(v) nucleic acid sequence which is an alternative splice variant of the nucleic acid sequences according to (i) to (iv);

The present invention also encompasses plants obtainable by the methods according to the present invention. The present invention therefore provides plants obtainable by the methods according to the present invention, which plants have modified growth and development and which plants have altered metallothionein activity and/or altered expression of a nucleic acid sequence encoding a metallothionein. Preferably, the plants are transgenic plants comprising an introduced nucleic acid sequence encoding a metallothionein and having increased yield and/or biomass, characterized in that the transgenic plant has been selected for modulated expression of a nucleic acid sequence encoding a metallothionein and/or modulated activity of a metallothionein. Further preferably, the transgenic plant has been selected for increased expression of a nucleic acid encoding metallothionein.

According to a third embodiment of the present invention, there is provided a method for the production of transgenic plants having modified growth characteristics, comprising introduction and expression in a plant of a nucleic acid molecule encoding a metallothionein as described above.

More specifically, the present invention provides a method for the production of transgenic plants having modified growth characteristics, which method comprises:
(i) introducing into a plant or plant cell a nucleic acid sequence or a portion thereof encoding metallothionein or a homologue, derivative or active fragment thereof;
(ii) regenerating and/or growing a plant from a transgenic plant cell.

The protein itself and/or the nucleic acid itself may be introduced directly into a plant cell or into the plant itself (including introduction into a tissue, organ or any other part of the plant). According to a preferred feature of the present invention, the nucleic acid sequence is preferably introduced into a plant by transformation. The nucleic acid sequence is preferably as represented by SEQ ID NO: 1 or a portion thereof or sequences capable of hybridising therewith, or is a nucleic acid sequence encoding an amino acid sequence represented by SEQ ID NO: 2 or a homologue, derivative or active fragment thereof. Alternatively, the nucleic add sequence is as represented by SEQ ID NO: 3 or a portion thereof or sequences capable of hybridising with any of the aforementioned sequences. The amino acid sequence may alternatively be a sequence as represented by SEQ ID NO: 4 or by homologues, derivatives or active fragments thereof.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell can then be used to regenerate a transformed plant in a manner known to persons skilled in the art.

Transformation of a plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., 1882, Nature 296, 72-74; Negrutiu I. et al., June 1987, Plant Mol. Biol. 8, 363-373); electroporation of protoplasts (Shillito R. D. et al., 1985 Bio/Technol 3, 1099-1102); microinjection into plant material (Crossway A. et al., 1986, Mol. Gen Genet 202, 179-185); DNA or RNA-coated particle bombardment (Klein T. M. et al., 1987, Nature 327, 70) infection with (non-integrative) viruses and the like. Transgenic rice plants expressing an metallothionein encoding gene are preferably produced via Agrobacterium-mediated transformation using any of the well known methods for rice transformation, such as described in any of the following: published European patent application EP 1198985 A1, Aldemita and Hodges (Planta, 199, 612-617, 1996); Chan et al. (Plant Mol. Biol. 22 (3) 491-506, 1993), Hiei et al. (Plant J. 6 (2) 271-282, 1994), which disclosures are incorporated by reference herein as if fully set forth. In the case of corn transformation, the preferred method is as described in either Ishida et al. (Nat. Biotechnol. 1996 June; 14(6): 745-50) or Frame et al. (Plant Physiol. 2002 May; 129(1): 13-22), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The present invention thus also encompasses transgenic plant comprising an introduced nucleic acid encoding metallothionein and having modified growth and development as defined above, characterised in the modified growth and development is the consequence of modulated expression of a nucleic acid encoding a metallothionein. The transgenic plants are furthermore selected modulated expression of a nucleic acid encoding a metallothionein. Preferably, the transgenic plants are selected for increased expression of a nucleic acid encoding metallothionein.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host cells containing an isolated nucleic acid molecule encoding a protein capable of modulating a metallothionein, preferably wherein the protein is a metallothionein. Preferred host cells according to the invention are plant cells. Therefore, the invention also encompasses host cells or transgenic plants having altered growth characteristics, characterized in that said host cell or transgenic plant has modulated expression of a nucleic acid sequence encoding a metallothionein and/or modulated activity and/or level of a metallothionein. The invention also extends to harvestable parts of a plant such as but not limited to seeds, leaves, fruits, flowers, stem cultures, rhizomes, tubers and bulbs.

The term “plant” as used herein encompasses whole plants, ancestors and progeny of the plants and plant parts, including seeds, shoots, stems, roots (including tubers), and plant cells, tissues and organs. The term “plant” also therefore encompasses suspension cultures, embryos, meristematic regions, callus tissue, leaves, seeds, roots, shoots, gametophytes, sporophytes, pollen, and microspores. Plants that are particularly useful in the methods of the invention include algae, ferns and all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Clyptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incarnata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Omithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vericillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees and algae amongst others. According to a preferred feature of the present invention, the plant is a crop plant comprising soybean, sunflower, canola, alfalfa, rapeseed or cotton. Further preferably, the plant according to the present invention is a monocotyledonous plant such as sugarcane, most preferably a cereal, such as rice, maize, wheat, millet, barley, oats, sorghum.

However, it is envisaged that the methods of the present invention can be applied to a wide variety of plants, since the high degree of sequence conservation among the known eukaryotic metallothionein homologues suggests an equally conserved function in cellular metabolism.

The present invention also relates to use of a metallothionein protein or to the use of a nucleic acid sequence encoding a metallothionein in modifying the growth and development of plants, preferably in modifying the growth characteristics of plants, preferably in Increasing yield and/or biomass and modifying plant architecture, provided that said modified growth and development is not increased metal accumulation or increased tolerance or resistance to abiotic stress. The nucleic acid sequence is preferably as represented by SEQ ID NO: 1 or a portion thereof or sequences capable of hybridising therewith or is an amino acid sequence represented by SEQ ID NO: 2 or a homologue, derivative or active fragment thereof. Preferably, the increase in yield and/or biomass is an increase in seed yield, more preferably the increase in seed yield comprises one or more of total number of seeds, total weight of seeds and a higher number of primary panicles.

The present invention also relates to use of a nucleic acid sequence encoding a metallothionein and homologues, derivatives and active fragments thereof and to use of the metallothionein itself and to homologues, derivatives and active fragments thereof as a growth regulator. The nucleic acid sequences hereinbefore described (and portions of the same and sequences capable of hybridising with the same) and the amino acid sequences hereinbefore described (and homologues, derivatives and active fragments of the same) are useful in modifying the growth characteristics of plants, as hereinbefore described. The sequences would therefore find use as growth regulators, to stimulate or inhibit plant growth. Therefore, the present invention provides a composition comprising a protein represented by SEQ ID NO 2 or a homologue, derivative or active fragment thereof for use in increasing yield and/or biomass of plants. The present invention furthermore provides a composition comprising a nucleic acid as represented by SEQ ID NO 1 or a portion thereof or a sequence hybridising therewith for use in increasing yield and/or biomass of plants. The present invention also provides a composition comprising a protein represented by any of the aforementioned amino acid sequences or homologues, derivatives or active fragments thereof for the use as a growth regulator.

Conversely, the sequences according to the present invention may also be interesting targets for agrochemical compounds, such as herbicides or growth stimulators. Accordingly, the present invention encompasses use of the aforementioned nucleic acid sequences (or a portion of the same or sequences capable of hybridising with the same) or an amino acid sequence as hereinbefore described (or homologues, derivatives and active fragments of the same) as targets for an agrochemical compound, such as a herbicide or a growth stimulator.

The methods according to the present invention result in plants having modified growth characteristics, as described hereinbefore. These advantageous growth characteristics may also be combined with other economically advantageous traits, such as further yield-enhancing traits, tolerance to various stresses, traits modifying various architectural features and/or biochemical and/or physiological features. The methods according to the present invention may also be practised by co-expression of a gene encoding a metallothionein protein in a plant with at least one other gene that cooperates with the gene encoding a metallothionein protein.

### DESCRIPTION OF FIGURES

The present invention will now be described with reference to the following figures in which:

FIG. 1: Alignment of various plant metallothionein sequences (Cobbett and Goldsbrough, 2002). Conserved cysteines in the N-terminal and C-terminal regions are indicated with an asterisk. Abbreviabons: At, Arabidopsis vulgaris, Bn, Brassica napus, Os, Oryza sativa, Ps Pisum sativum, Ms, Medicago saliva, Bo, Brassica oleracea, Ph, Petunia hybrida, Sv, Silene vulgaris, Ma, Musa acuminata, Ad, Actinidia deliciosa, Gh, Gossipium hirsutum, Pg, Picea glauca, Zm, Zea mays, Ta, Triticum aestivum.

FIG. 2: Schematic presentation of the entry done p33, containing CDS1585 within the AttL1 and AttL2 sites for Gateway® cloning in the pDONR201 backbone. CDS1585 is the internal code for the Arabidopsis thaliana metallothionein-like AtMT2a coding sequence. This vector contains also a bacterial kanamycine-resistance cassette and a bacterial origin of replication.

FIG. 3: Binary vector for the expression in Oryza sativa of the Arabidopsis thaliana metallothionein-like AtMT2a gene (CDS1585) under the control of the rice GOS2 promoter (PRO0129). This vector contains a T-DNA derived from the Ti Plasmid, limited by a left border (LB repeat, LB Ti C58) and a right border (RB repeat, RB Ti C58)). From the left border to the right border, this T-DNA contains: a cassette for antibiotic selection of transformed plants; a cassette for visual screening of transformed plants; the PRO0129-CDS1585-zein and rbcS-deltaGA double terminator expression cassette (SEQ ID NO 7) for expression of the Arabidopsis thaliana metallothionein-like AtMT2a gene. This vector also contains an origin of replication from pBR322 for bacterial replication and a selectable marker (Spe/SmeR) for bacterial selection with spectinomycin and streptomycin.

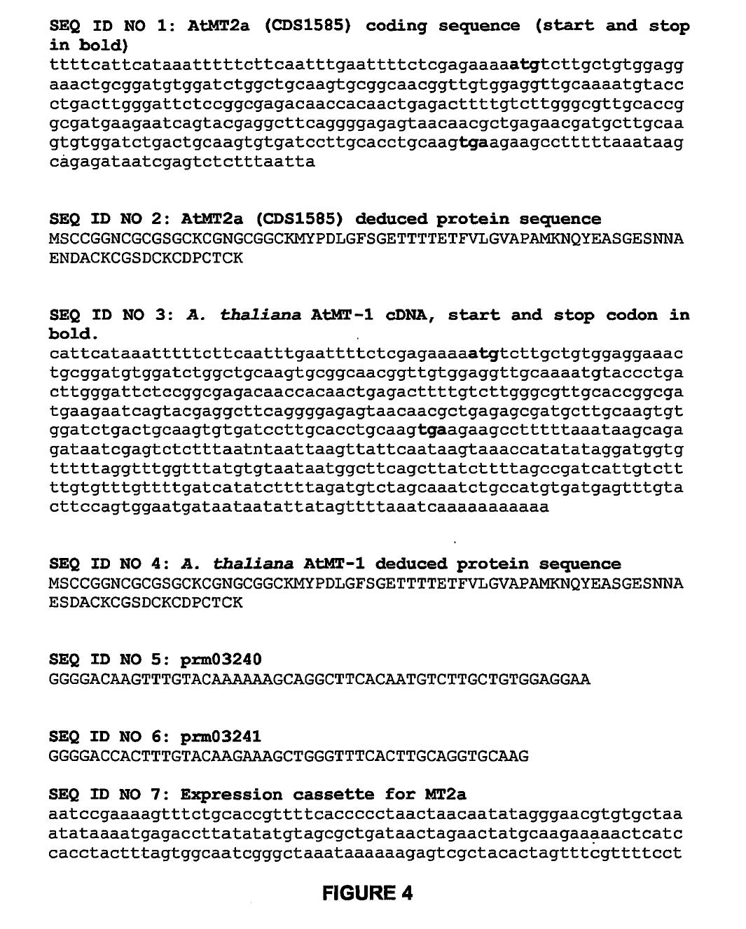

FIG. 4: Sequence listing

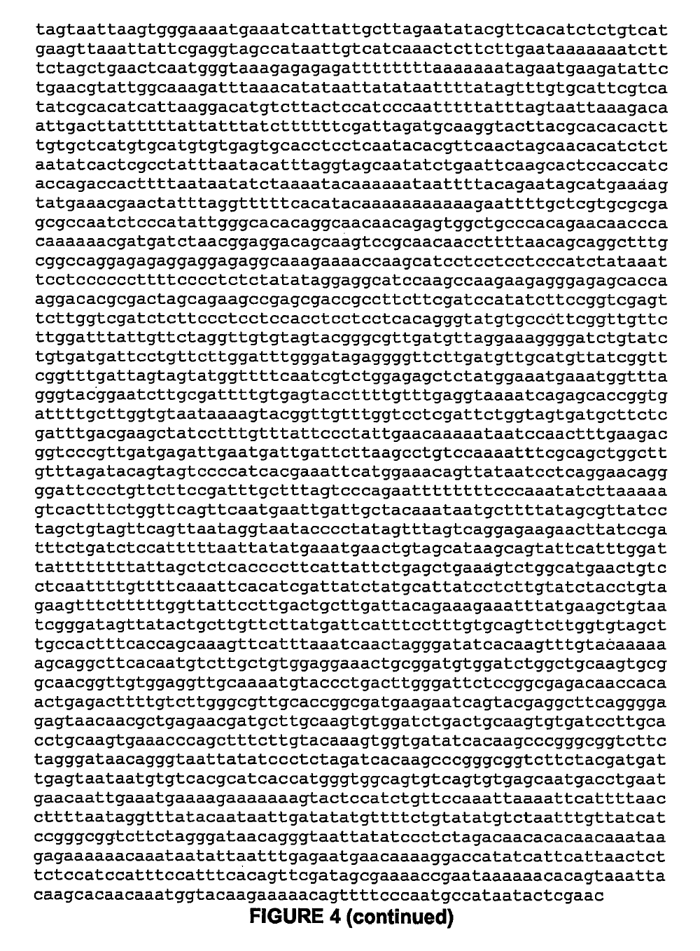

### EXAMPLE

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1984), Current Protocols in Molecular Biology, Current Protocols. Standard materials and methods for plant molecular work are described in Plant Molecular Biology Labfase (1993) by R. D. D. Croy, published by BIOS Scientific Publications Ltd (UK) and Blackwell Scientific Publications (UK).

### Example 1

### Cloning of CDS0851

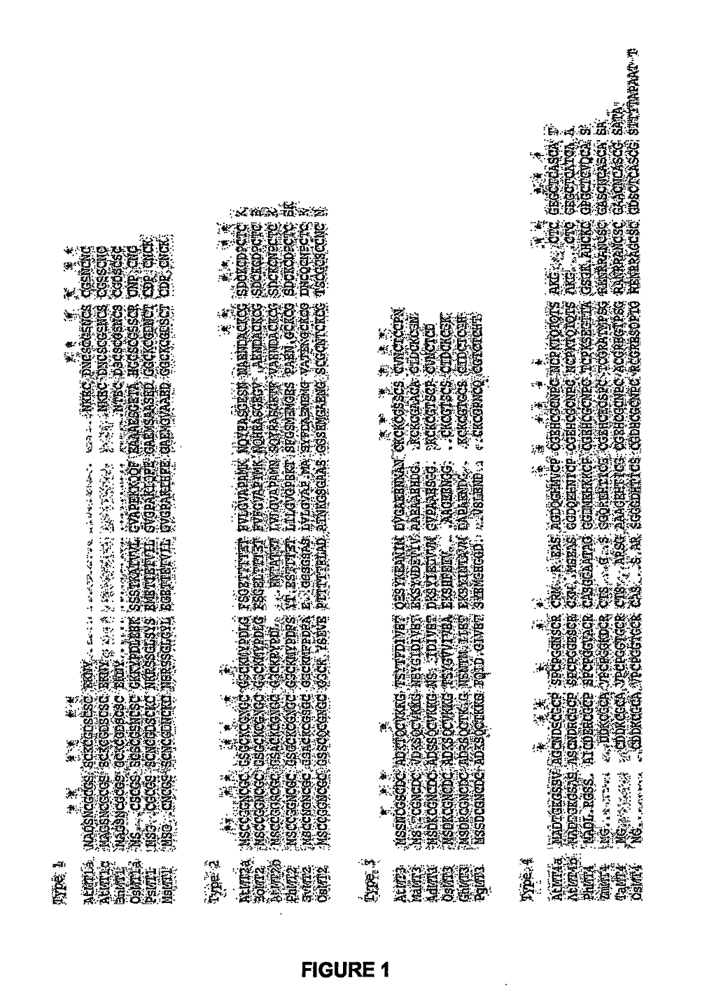

The Arabidopsis metallothionein coding sequence AtMT2a (CDS1585) was amplified by PCR using as template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb, and original number of clones was of 1.59×10⁷ cfu. The original titer was determined to be 9.6×10⁵ cfu/ml, and after a first amplification became 6×10¹¹ cfu/ml. After plasmid extraction, 200 ng of template was used in a 50 μl PCR mix. Primers prm03240 (SEQ ID NO 5) and prm03241 (SEQ ID NO 6), which include the AttB sites for Gateway recombination, were used for PCR amplification. PCR was performed using Hifi Taq DNA polymerase in standard conditions. A PCR fragment of 246 bp was amplified and purified also using standard methods. The first step of the Gateway procedure, the BP reaction, was then performed, during which the PCR fragment recombines In vivo with the pDONR201 plasmid to produce, according to the Gateway terminology, an “entry clone”, p33 (FIG. 1). Plasmid pDONR201 was purchased from Invitrogen, as part of the Gateway® technology.

### Example 2

### Vector Construction for Transformation with PRO0129-CDS1585 Cassette

The entry clone p33 was subsequently used in an LR reaction with p0640, a destination vector used for Oryza sativa transformation. This vector contains as functional elements within the T-DNA borders: a plant selectable marker; a screenable marker expression cassette; and a Gateway cassette intended for LR in vivo recombination with the sequence of interest already cloned in the entry done. A rice GOS2 promoter for constitutive expression (PRO0129) is located upstream of this Gateway cassette.

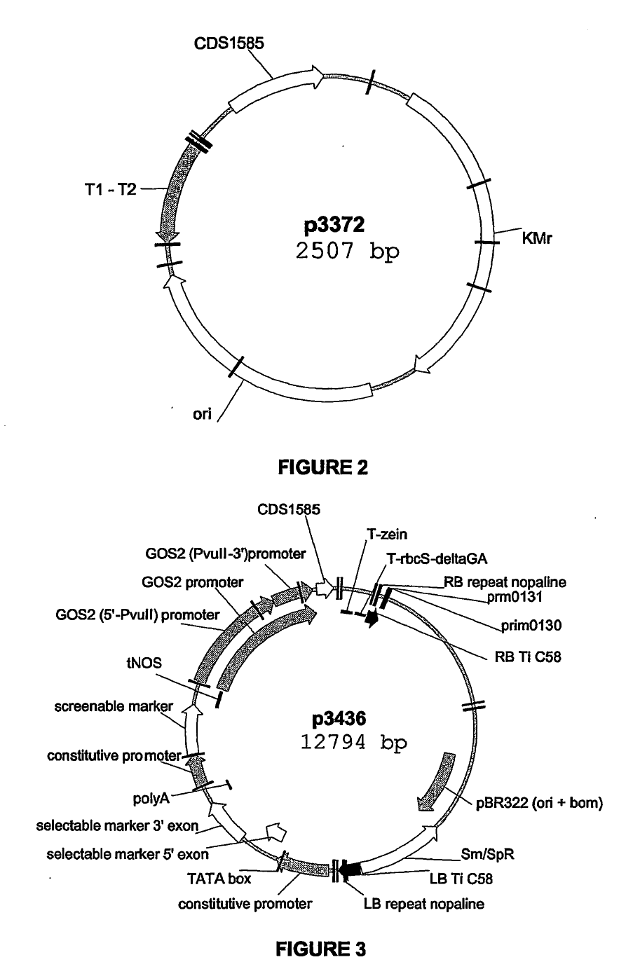

After the LR recombination step, the resulting expression vector p34 (FIG. 2) can be transformed into the Agrobacerium strain LBA4044 and subsequently to Oryza saliva plants.

### Example 3

### Transformation of Rice with PRO0129-CDS1585

Mature dry seeds of Oryza saliva japonica cultivar Nipponbare were dehusked. Sterilization was done by incubating the seeds for one minute in 70% ethanol, followed by 30 minutes in 0.2% HgCl₂ and by 6 washes of 15 minutes with sterile distilled water. The sterile seeds were then germinated on a medium containing 2,4-D (callus induction medium). After a 4-week incubation in the dark, embryogenic, scutellum-derived calli were excised and propagated on the same medium. Two weeks later, the calli were multiplied or propagated by subculture on the same medium for another 2 weeks. 3 days before co-cultivation, embryogenic callus pieces were sub-cultured on fresh medium to boost cell division activity. The Agrobacterium strain LBA4044 harbouring the binary vector p3076 was used for co-cultivation. The Agrobacterium strain was cultured for 3 days at 28° C. on AB medium with the appropriate antibiotics. The bacteria were then collected and suspended in liquid co-cultivation medium at an OD₆₀₀ of about 1. The suspension was transferred to a petri dish and the call were immersed in the suspension during 15 minutes. Next, the callus tissues were blotted dry on a filter paper, transferred to solidified co-cultivation medium and incubated for 3 days in the dark at 25° C.

Hereafter, co-cultivated callus was grown on 2,4-D-containing medium for 4 weeks in the dark at 28° C. in the presence of a selective agent at a suitable concentration. During this period, rapidly growing resistant callus islands developed. Upon transfer of this material to a regeneration medium and incubation in the light, the embryogenic potential was released and shoots developed in the next four to five weeks. Shoots were excised from the callus and incubated for 2 to 3 weeks on an auxin-containing medium from which they were transferred to soil. Hardened shoots were grown under high humidity and short days in a greenhouse. Finally seeds were harvested three to five months after transplanting. The method yielded single locus transformants at a rate of over 50% (Aldemita and Hodges, 1996, Chan et al., 1993, Hiei et al., 1994).

### Example 4

### Evaluation of Transgenic Rice Transformed with PRO0129-CDS1585

Approximately 15 to 20 independent T0 transformants were generated. The primary transformants were transferred from tissue culture chambers to a greenhouse for growing and harvest of T1 seed. Seven events of which the T1 progeny segregated 3:1 for presence/absence of the transgene were retained. For each of these events, 10 T1 seedlings containing the transgene (hetero- and homo-zygotes), and 10 T1 seedlings lacking the transgene (nullizygotes), were selected by visual marker screening. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of seed-related parameters described below.

These parameters were derived in an automated way from the digital images using image analysis software and were analysed statistically. A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test shows that the data are significant, than it is concluded that there is a “gene” effect, meaning that not only presence or the position of the gene is causing the effect. The threshold for significance for a true global gene effect is set at 5% probability level for the F test.

To check for an effect of the genes within an event, i.e., for a line-specific effect, a t-test was performed within each event using data sets from the transgenic plants and the corresponding null plants. “Null plants” or “Null segregants” or “Nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect In certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

Vegetative growth and seed yield were measured according to the methods as described above. The inventors surprisingly found that the total number and total weight of seeds and the number of primary panicles were increased in the rice plants transformed with the metallothionein gene when compared the control plants without the AtMt2a gene.

The data obtained in the first experiment were confirmed in a second experiment with T2 plants. Three lines that had the correct expression pattern were selected for further analysis. Seed batches from the positive plants (both hetero- and homozygotes) in T₁, were screened by monitoring marker expression. For each chosen event, the heterozygote seed batches were then retained for T2 evaluation. Within each seed batch an equal number of positive and negative plants were grown in the greenhouse for evaluation.

A total number of 120 Mt2a transformed plants were evaluated in the T2 generation, that is 40 plants per event of which 20 positives for the transgene, and 20 negatives.

Since two experiments with overlapping events had been carried out, a combined analysis was performed in addition to the above described analysis. This is useful to check consistency of the effects over the two experiments, and if this is the case, to accumulate evidence from both experiments in order to increase confidence in the conclusion. The method used was a mixed-model approach that took into account the multilevel structure of the data (i.e. experiment-event-segregants). P-values were obtained by comparing likelihood ratio test to chi square distributions.

### Example 5

### Results of the Evaluation of Transgenic Plants Transformed with PRO0129-CDS1585

Upon analysis of the seeds as described above, the inventors found that plants transformed with the Mt2a gene construct had a higher number of primary panicles, a higher total number of seeds and a higher total weight of seeds compared to plants lacking the Mt2a transgene. Positive results obtained for plants in the T1 generation were again obtained in the T2 generation. Not only individual transgenic lines scored significantly better than the corresponding nullizygous control lines, but there was also a significant positive overall effect when all plants of all tested T2 events were evaluated, strongly Indicating a global gene effect. An overview of the data is given in Table 2.

TABLE 2

| | T1 plants | | T2 plants | | Combined |
|---|---|---|---|---|---|
| | % dif- | | % dif- | | analysis |
| parameter | ference | p-value | ference | p-value | p-value |
| Total | 14 | 0.0404 | 16 | 0.0184 | 0.0003 |
| number | | | | | |
| of seeds | | | | | |
| Total | 9 | 0.2092 | 24 | 0.0091 | 0.0017 |
| weight | | | | | |
| of seeds | | | | | |
| Number | 16 | 0.0958 | 13 | 0.0907 | 0.0002 |
| of primary | | | | | |
| panicles | | | | | |

The % increase presents the average increase for all tested events. The p-values for the T1 and T2 plants stand for the p-value derived from the F-test and the p-values for the combined analysis were obtained by comparing likelihood ratio test to chi square distributions.

Total Number of Seeds

Total seed number per plant was measured by counting the number of husks harvested from a plant. Transgenic T1 lines showed an overall increase in total seed number of 14%, which increase was significant. This increase was confirmed in experiments with T2 plants, where a significant increase of 16% was measured. The combined analysis of T1 and T2 plants showed a global gene effect (p-value of 0.0003).

Total Seed Yield

The total seed yield (total weight of seeds) per plant was measured by weighing all filled husks harvested from a plant. On average, the increase in seed yield for T1 plants was 9%. These results were also observed in the T2 generation. The 3 tested lines had an average yield increase of 24%. This mean increase was statistically significant (p-value of 0.0091) and the combined analysis of the T1 and T2 plants showed there was a global gene effect (p-value of 0.0017).

Number of Primary Panicles

The tallest panicle and all the panicles that overlapped with the tallest panicle when aligned vertically were considered as primary panicles, and counted manually. There was an overall effect of the transgene on the number of panicles: the increase for the T1 plants was 16% and 13% for the T2 plants. These Increases were significantly as evidenced by the p-values (respectively 0.0958 and 0.0907). The combined analysis demonstrated a global gene effect (p-value 0.0002).

A variation between the different transformation events (different plant events each transformed with the AtMT2a gene) was observed. It is well known to persons skilled in the art that the expression of transgenes in plants, and hence also the phenotypic effect due to expression of such transgene, can differ among different independently obtained transgenic lines and progeny thereof. The transgenes present in different independently obtained transgenic plants differ from each other by the chromosomal insertion locus as well as by the number of transgene copies inserted in that locus and the configuration of those transgene copies in that locus. Differences in expression levels can be ascribed to influence from the chromosomal context of the transgene (the so-called position effect) or from silencing mechanisms triggered by certain transgene configurations (e.g. inwards facing tandem insertions of transgenes are prone to silencing at the transcriptional or post-transcriptional level). Despite this variability, the analysis of the T1 and T2 plants and the combined analysis showed that the observed increases in yield are caused by a true global gene effect.

### Example 6

### Modifying Kernel Yield in Zea mays

The invention described herein can also be used in maize. To this aim, the Mt2a gene, or the maize orthologue thereof is cloned under control of a suitable promoter like the rice GOS2 promoter or another constitutive promoter in a plant transformation vector suited for Agrobacterium-mediated corn transformation. Such vectors and methods for corn transformation have been described in literature (EP0604662, EP0672752, EP0971578, EP0955371, EP0558676, Ishida et al. 1996; Frame et al., 2002). Transgenic plants made by these methods are grown in the greenhouse for T1 seed production. Heritability is checked by progeny segregation analysis. Copy number of the transgene is checked by quantitative real-time PCR and/or Southern blot analysis. Expression levels of the transgene are determined by reverse PCR and/or Northern analysis. Transgenic lines with single copy insertions of the transgene and with varying levels of transgene expression are selected for T2 seed production through selfing or for crossing to different germplasm. Progeny seeds are germinated and grown in the field or in the greenhouse in conditions well adapted for maize (16:8 hr photoperiod, 26-28° C. daytime and 22-24° C. night time temperature) as well under water-deficient, nitrogen-deficient, and excess NaCl conditions. In the case of selfing, null segregants from the same parental line, as well as wild type plants of the same cultivar are used as controls. In the case of crossing, transgenics, null segregants and wild type plants of the same cultivar are crossed to a chosen parent and F1 plants from the transgenic cross are compared to F1 plants from the null segregant and the wild type crosses. The progeny plants resulting from the selfing or the crosses are evaluated on different biomass and growth parameters, including plant height, stem thickness, number of leaves, total above ground area, leaf greenness, time to maturity, flowering time, ear number, harvesting time. The seeds of these lines are also checked on various parameters, such as grain size, total grain yield per plant, and grain quality (starch content, protein content and oil content). Lines that are most significantly improved versus the controls for any of the above-mentioned parameters are selected for further field testing and marker-assisted breeding, with the objective of transferring the field-validated transgenic traits into commercial germplasm. Methods for testing maize for growth and yield-related parameters in the field are well established in the art, as are techniques for introgressing specific loci (such as transgene containing loci) from one germplasm into another.

1. Method for modifying plant growth and development, comprising introducing a genetic modification in said plant and selecting for modulated expression in said plant of a nucleic acid encoding a metallothionein protein, provided that said modified growth and development is not increased metal accumulation or increased tolerance or resistance to abiotic stress.

2. Method according to claim 1, wherein said modified plant growth and development is increased yield, preferably an increase of biomass and/or seed yield, when compared to corresponding wild type plants.

3. Method according to claim 2, wherein said increased seed yield comprises increased total number of seeds and/or increased total weight of seeds, when compared to corresponding wild type plants.

4. Method according to claim 1, wherein said modulated expression is increased expression.

5. Method according to claim 1, wherein said genetic modification comprises introducing an isolated nucleic acid encoding a metallothionein protein into a plant.

6. Method according to claim 1, wherein said nucleic acid encoding a metallothionein protein encodes a type 2 metallothionein.

7. Method according to claim 6, wherein said nucleic acid is derived from a plant, preferably from a dicotyledonous plant, more preferably from the family Brassicaceae, most preferably the nucleic acid sequence is from Arabidopsis thaliana.

8. Method according to claim 5, wherein said nucleic acid sequence and said proteins include variants chosen from:
(i) a nucleic acid as represented by SEQ ID NO: 1 or encoding a metallothionein protein as represented by SEQ ID NO: 2;
(ii) an alternative splice variant of a nucleic acid sequence encoding a metallothionein protein or wherein said metallothionein protein is encoded by a splice variant;
(iii) an allelic variant of a nucleic acid sequence encoding a metallothionein protein or wherein said metallothionein protein is encoded by an allelic variant;
(iv) a nucleic acid encoding a functional portion of a metallothionein protein;
(v) a metallothionein protein as represented by SEQ ID NO: 2
(vi) homologues, derivatives and active fragments of a metallothionein protein.

9. Method according to claim 5, wherein expression of said nucleic acid encoding a metallothionein is driven by a constitutive promoter, preferably the rice GOS2 promoter.

10. Plants obtainable by a method according to claim 1.

11. A construct comprising:
(i) a nucleic acid sequence encoding a metallothionein;
(ii) a GOS2 promoter capable of driving expression of the nucleic acid sequence of (i); and optionally
(iii) a transcription termination sequence.

12. Construct according to claim 11, wherein said nucleic acid sequence encoding a metallothionein is chosen from the group comprising:
(i) a nucleic acid sequence represented by SEQ ID NO: 1 or the complement strand thereof;
(ii) a nucleic acid sequence encoding an amino acid sequence represented by SEQ ID NO: 2 or homologues, derivatives or active fragments thereof;
(iii) a nucleic acid sequence according to (i) to (ii) above which is degenerated as a results of the genetic code;
(iv) nucleic acid sequence which is an allelic variant of the nucleic acid sequences according to (i) to (iii);
(v) nucleic acid sequence which is an alternative splice variant of the nucleic acid sequences according to (i) to (iv);

13. Construct comprising the expression cassette as presented in SEQ ID NO 7.

14. Method for the production of a transgenic plant having modified growth and development according to claim 1, which method comprises:
(i) introducing into a plant or plant cell a nucleic acid sequence or a portion thereof encoding a metallothionein or a homologue, derivative or active fragment thereof;
(ii) regenerating and/or growing a plant from a transgenic plant cell.

15. Transgenic plant comprising an introduced nucleic acid encoding metallothionein and having modified growth and development according to claim 1, characterised in that the modified growth and development is the consequence of modulated expression of a nucleic acid encoding a metallothionein.

16. Transgenic plant according to claim 15, selected for increased expression of a nucleic acid encoding metallothionein.

17. Transgenic plant according to claim 15, wherein said plant is a crop plant comprising soybean, sunflower, canola, alfalfa, rapeseed or cotton, preferably, the plant according to the present invention is a monocotyledonous plant such as sugarcane, most preferably a cereal, such as rice, maize, wheat, millet, barley, oats, sorghum.

18. Transgenic plant cells, transgenic plants or transgenic plant parts, including harvestable parts, propagules, seeds or transgenic progeny of a plant according to claim 15.

19. Use of a nucleic acid sequence encoding a metallothionein or encoding homologues, derivatives or active fragments thereof in modifying the growth characteristics of plants, provided that said modified growth and development is not increased metal accumulation or increased tolerance or resistance to abiotic stress.

20. Use of a metallothionein protein and homologues, derivatives and active fragments thereof in modifying the growth characteristics of plants, provided that said modified growth and development is not increased metal accumulation or increased tolerance or resistance to abiotic stress.

21. Use according to claim 19, wherein said modified growth and development is increased yield and/or increased biomass, preferably increased seed yield, when compared to corresponding wild type plants.

22. Use according to claim 21, wherein said increased seed yield comprises increased total number of seeds and/or increased total weight of seeds, when compared to corresponding wild type plants.

23. A composition comprising a nucleic acid sequence as represented by SEQ ID NO: 1 or a portion thereof or a sequence represented by SEQ ID NO: 2 or homologues, derivatives and active fragments thereof for modifying plant growth and development, preferably for increasing yield and/or biomass of a plant, more preferably for increasing seed yield, provided that said modified growth and development is not increased metal accumulation or increased tolerance or resistance to abiotic stress.

24. Use of a nucleic acid sequence as represented by SEQ ID NO: 1 or a portion thereof or a sequence represented by SEQ ID NO: 2 or homologues, derivatives and active fragments thereof as a growth regulator.
